SEQUENCE LISTING (2 sequences)

SEQ ID NO: 1 - DNA, 27 bp, Homo sapiens
ttcaaatgagattgtgggaaaattgct

SEQ ID NO: 2 - DNA, 24 bp, Homo sapiens
agatcatctctgcctgagtatctt

(19) United States
(12) Patent Application Publication
(10) Pub. No.: US 2003/0044829 A1
(43) Pub. Date: Mar. 6, 2003

(54) Methods for detecting and monitoring COX-2 RNA in plasma and serum

(76) Inventor: Michael S. Kopreski, Long Valley, NJ (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/178,290

(22) Filed: Jun. 24, 2002

Related U.S. Application Data
(60) Provisional application No. 60/300,751, filed on Jun. 25, 2001.

(51) Int. Cl. C12Q 1/68; C12P 19/34

(52) U.S. Cl. 435/6; 435/91.2

(57) ABSTRACT

This invention provides methods for detecting or inferring the presence of malignant or premalignant cells in a human wherein the malignant or premalignant cells express COX-2. The methods of the invention detect extracellular COX-2 RNA in blood, plasma, serum, and other bodily fluids. The inventive methods are useful for aiding detection, diagnosis, monitoring, treatment, or evaluation of neoplastic disease, and for identifying individuals for whom COX-2 directed therapies would be beneficial.

This application claims priority to U.S. Provisional Patent Application Serial No. 60/300,751, filed Jun. 25, 2001, the disclosure of which is specifically incorporated by reference herein.

### BACKGROUND OF THE INVENTION

This invention relates to methods for detecting and monitoring cyclooxygenase-2 RNA (COX-2 RNA) in bodily fluids such as blood plasma, serum, and other bodily fluids. The invention particularly enables detection and monitoring of extracellular COX-2 RNA in plasma, serum, and other bodily fluids, such as COX-2 RNA within apoptotic bodies or fragments or vesicles present in the bodily fluid. The invention provides uses and applications for said detection and monitoring, particularly as applied to cancer management.

COX-2 is an inducible enzyme that converts arachidonic acids to prostaglandins, and is expressed in many malignant, premalignant, and non-malignant tissues. COX-2 also plays a major role in the development of premalignant and malignant tumors, being particularly associated with cells which become invasive Since ribonucleic acid (RNA) is essential for producing COX-2 protein, detection and monitoring of COX-2 RNA provides a method for assessing and monitoring COX-2 gene expression.

Several reports have indicated that certain RNA species may be detected in plasma or serum (Kopreski et al., 1999, Clin. Cancer Res. 5: 1961-1965; Chen et al., 2000, Clin. Cancer Res. 6: 3823-3826). Co-owned U.S. Pat. No. 6,329,179B1, incorporated herein by reference in its entirety, provides methods for detecting tumor-associated RNA in bodily fluids such as blood plasma and serum. However, whether COX-2 RNA was detectable in plasma or serum, and thereby applications from such detection, were not known in the art prior to this invention. Others in the art have indicated that not all RNA species may be readily detectable in plasma or serum (Hasselmann et al., 2001, Oncology Reports 8: 115-118; Komeda et al., 1995, Cancer 75: 2214-9; Pfleiderer et al., 1995, Int. J. Cancer 64: 135-139).

Because COX-2 RNA is expressed in several disease states and conditions including cancer, there is a newly-appreciated need in the art to identify premalignant or malignant states in an animal, most preferably a human, and further to identify premalignant or malignant conditions that overexpress COX-2 RNA, by detecting COX-2 RNA in bodily fluids such as blood plasma or serum.

### SUMMARY OF THE INVENTION

The present invention provides methods for evaluating an animal, most preferably a human, for premalignant or malignant states, disorders or conditions by detecting COX-2 mRNA in bodily fluids, preferably blood and most preferably blood plasma and serum as well as in other bodily fluids, preferably urine, effusions, ascites, saliva, cerebrospinal fluid, cervical, vaginal, and endometrial secretions, gastrointestinal secretions, bronchial secretions, breast fluid, and associated tissue washings and lavages.

The invention provides methods of amplifying and detecting extracellular COX-2 RNA from a bodily fluid. In a preferred embodiment, the present invention provides methods for detecting extracellular COX-2 RNA in blood or a blood fraction, including plasma and serum, or in other bodily fluids. As provided herein, the method comprises the steps of extracting RNA from blood, plasma, serum, or other bodily fluid, in vitro amplifying or signal amplifying COX-2 mRNA or its cDNA, and detecting the amplified product or amplified signal of COX-2 mRNA or its cDNA.

In a first aspect of this embodiment, the present invention provides methods for detecting extracellular COX-2 RNA in blood or blood fractions, including plasma and serum, in a human or animal. Said methods are useful for detecting, diagnosing, monitoring, treating and evaluating various proliferative disorders, particularly stages of neoplastic disease, including premalignancy, early cancer, non-invasive cancer, carcinoma in-situ, invasive cancer and advanced cancer, as well as benign neoplasm. In this aspect, the method comprises the steps of extracting RNA from blood or blood plasma or serum, in vitro amplifying or signal amplifying said COX-2 RNA comprising the extracted RNA either qualitatively or quantitatively, and detecting the amplified product or signal of COX-2 RNA or its cDNA.

The invention in a second aspect provides methods for detecting extracellular COX-2 RNA in any bodily fluid. Preferably, said bodily fluid is whole blood, blood plasma, serum, urine, effusions, ascitic fluid, saliva, cerebrospinal fluid, cervical secretions, vaginal secretions, endometrial secretions, gastrointestinal secretions, bronchial secretions including sputum, secretions or washings from the breast, or other associated tissue washings or lavages from a human or animal. In this aspect, the method comprises the steps of extracting RNA from the bodily fluid, in vitro amplifying or signal amplifying COX-2 RNA comprising a fraction of the extracted RNA, or preferably the corresponding cDNA into which the RNA is converted, in a qualitative or quantitative fashion, and detecting the amplified product or signal of COX-2 RNA or cDNA. In these embodiments, the inventive methods are particularly advantageous for detecting, diagnosing, monitoring, treating or evaluating various proliferative disorders, particularly stages of neoplastic disease, including premalignancy, early cancer, non-invasive cancer, carcinoma-in-situ, invasive cancer and advanced cancer, as well as benign neoplasm. In additional aspects, the method is further applied for evaluation of non-neoplastic diseases, including arthritis and inflammatory diseases.

The methods of the invention are additionally useful for identifying COX-2 RNA over-expressing cells or tissue in an animal, most preferably a human. In these embodiments, detection of an in vitro amplified product of COX-2 RNA derived from a non-cellular fraction of a bodily fluid using the inventive methods is used to evaluate for COX-2 RNA over-expressing cells or tissue in an animal, most preferably a human.

The invention provides primers useful in the efficient amplification of COX-2 mRNA or cDNA from bodily fluid, most preferably blood plasma or serum.

The invention further provides a diagnostic kit for detecting COX-2 RNA in bodily fluid, preferably blood plasma or serum, wherein the kit comprises primers, probes or both primers and probes for amplifying and detecting extracellular COX-2 RNA or cDNA derived therefrom, and may further include reagents for the extraction of RNA from the bodily fluid, or for reverse transcription, amplification, or detection of the COX-2 RNA or cDNA derived therefrom.

In preferred embodiments of the inventive methods, COX-2 RNA is extracted from whole blood, blood plasma or serum, or other bodily fluids using an extraction method such as gelatin extraction method; silica, glass bead, or diatom extraction method; guanidinium thiocyanate acid-phenol based extraction methods; guanidinium thiocyanate acid based extraction methods; methods using centrifugation through cesium chloride or similar gradients; phenol-chloroform based extraction methods; or other commercially available RNA extraction methods. Extraction may further be performed using probes that specifically hybridize to COX-2 RNA.

In preferred embodiments of the inventive methods, COX-2 RNA or cDNA derived therefrom is amplified using an amplification method such as polymerase chain reaction (PCR); reverse transcriptase polymerase chain reaction (RT-PCR); ligase chain reaction; DNA signal amplification; amplifiable RNA reporters; Q-beta replication; transcription-based amplification; isothermal nucleic acid sequence based amplification; self-sustained sequence replication assays; boomerang DNA amplification; strand displacement activation; cycling probe technology; or any combination or variation thereof.

In preferred embodiments of the inventive methods, detecting an amplification product of COX-2 RNA or COX-2 cDNA is accomplished using a detection method such as gel electrophoresis; capillary electrophoresis; conventional enzyme-linked immunosorbent assay (ELISA) or modifications thereof, such as amplification using biotinylated or otherwise modified primers; nucleic acid hybridization using specific, detectably-labeled probes, such as fluorescent-, radioisotope-, or chromogenically-labeled probe; laser-induced fluorescence; Northern blot analysis; Southern blot analysis; electrochemiluminescence; reverse dot blot detection; and high-performance liquid chromatography.

In particularly preferred embodiments of the inventive methods, COX-2 RNA is converted to cDNA using reverse transcriptase following extraction of RNA from a bodily fluid and prior to amplification.

In particularly preferred embodiments, extracellular COX-2 RNA extracted from blood plasma or serum, or its corresponding cDNA derived therefrom, is hybridized to a primer or probe specific for COX-2 RNA or its corresponding cDNA.

In particularly preferred embodiments, extracellular COX-2 RNA extracted from a non-cellular fraction of a bodily fluid, or its corresponding cDNA derived therefrom, is hybridized to a primer or probe specific for COX-2 RNA or its corresponding cDNA.

The methods of the invention are advantageously used for providing a diagnosis or prognosis of, or as a predictive indicator for determining a risk for an animal, most preferably a human, for developing a proliferative, premalignant, neoplastic or malignant disease comprising or characterized by the existence of cells expressing COX-2 RNA. The methods of the invention are particularly useful for providing a diagnosis for identifying humans at risk for developing or who have developed malignancy or premalignancy. Most preferably, the malignant or premalignant diseases, conditions or disorders advantageously detected or diagnosed using the methods of the invention are breast, prostate, ovarian, lung, cervical, colorectal, gastric, hepatocellular, pancreatic, bladder, endometrial, kidney, skin, and esophageal cancers, and premalignancies and carcinoma in-situ such as prostatic intraepithelial neoplasia (PIN), cervical dysplasia, cervical intraepithelial neoplasia (CIN), bronchial dysplasia, a typical hyperplasia of the breast, ductal carcinoma in-situ (DCIS), colorectal adenoma, a typical endometrial hyperplasia, and Barrett's esophagus.

In certain preferred embodiments of the methods of the invention, COX-2 RNA or cDNA derived therefrom is amplified in a quantitative manner, thereby enabling the quantitative comparison of COX-2 RNA present in a bodily fluid such as blood plasma or serum from an animal, most preferably a human. In these embodiments, the amount of extracellular COX-2 RNA detected in an individual are compared with a range of amounts of extracellular COX-2 RNA detected in said bodily fluid in populations of animals known to have a premalignant, neoplastic, or malignant disease, most preferably a particular premalignant, neoplastic, or malignant disease. Additionally, the amount of extracellular COX-2 RNA detected in an individual is compared with a range of amounts of extracellular COX-2 RNA detected in said bodily fluid in populations of humans or animals known to be free from a premalignant, neoplastic, or malignant disease. In one aspect of this embodiment, a risk for a premalignant or malignant disease is determined. In a second aspect of this embodiment, an individual having COX-2 RNA over-expressing cells or tissue is identified. In a third aspect of this embodiment, individuals who are unlikely to benefit from a COX-2 inhibitor therapeutic agent are identified.

The methods of the invention further provide ways to identify individuals having a COX-2 expressing malignancy or premalignancy, thereby permitting rational, informed treatment options to be used for making therapeutic decisions, and for monitoring response to treatment. In particular, the methods of the invention are useful in identifying individuals having a premalignancy or malignancy that might benefit from a COX-2-directed therapy such as administration of a therapeutically-effective amount of a COX-2 inhibitor drug, either alone or administered with therapeutically-effective amounts of other chemotherapeutic or anticancer drugs. The methods of the invention are further advantageous for monitoring the response of an individual to a COX-2 inhibitor drug, and thereby provide a prognostic indicator of therapeutic response.

Another advantageous use for the methods of the invention is to provide a marker for assessing the adequacy of anticancer therapy, including surgical intervention, chemotherapy, or radiation therapy, administered preventively or palliatively, or for determining whether additional or more advanced therapy is required. The invention therefore provides methods for developing a prognosis in such patients.

The methods of the invention also allows identification or analysis of COX-2 RNA, either qualitatively or quantitatively, in the blood or other bodily fluid of an individual, most preferably a human who has completed therapy, as an early indicator of relapsed cancer, impending relapse, or treatment failure.

Specific preferred embodiments of the present invention will become evident from the following more detailed description of certain preferred embodiments and the claims.

### DETAILED DESCRIPTION OF THE INVENTION

The invention provides methods for detecting extracellular COX-2 RNA in bodily fluids in an animal, most preferably a human, and thereby enabling the detection and monitoring of cancerous or precancerous conditions characterized by cells that express COX-2 in the human or animal. The practice of the methods of the invention advantageously permits individuals having said conditions to be identified or selected.

In preferred embodiments of the methods of the invention, extracellular RNA containing COX-2 RNA is extracted from a bodily fluid. This extracted RNA is then amplified, either after conversion into cDNA or directly, using in vitro amplification methods in either a qualitative or quantitative manner using primers or probes specific for COX-2 RNA. The amplified product is then detected in either a qualitative or quantitative manner.

In the practice of the methods of the invention, extracellular COX-2 RNA may be extracted from any bodily fluid, including but not limited to whole blood, plasma, serum, urine, effusions, ascitic fluid, saliva, cerebrospinal fluid, cervical secretions, vaginal secretions, endometrial secretions, gastrointestinal secretions, bronchial secretions including sputum, breast fluid, or secretions or washings or lavages, using, for example, extraction methods described in co-owned U.S. Pat. No. 6,329,179B1, the entire disclosure of which is hereby incorporated by reference. In a preferred embodiment, the bodily fluid is either blood plasma or serum. It is preferred, but not required, that blood be processed soon after drawing, and preferably within three hours, as to minimize any nucleic acid degradation in the sample. In a preferred embodiment, blood is first collected by venipuncture and kept on ice until use. Preferably, within 30 minutes to one hour of drawing the blood, serum is separated by centrifugation, for example at 1100× g for 10 minutes at 4° C. When using plasma, the blood is not permitted to coagulate prior to separation of the cellular and acellular components. Serum or plasma can be frozen, for example at −70° C. after separation from the cellular portion of blood until further assayed. When using frozen blood plasma or serum, the frozen serum or plasma is rapidly thawed, for example in a 37° C. water bath, and RNA is extracted therefrom without delay, most preferably using a commercially-available kit (for example, Perfect RNA Total RNA Isolation Kit, obtained from Five Prime-Three Prime, Inc., Boulder, Colo.), according to the manufacturer's directions. Other methods of RNA extraction are further provided in co-owned U.S. Pat. No. 6,329,179B1, incorporated herein by reference in its entirety.

Following extraction of RNA from a bodily fluid, a fraction of which contains COX-2 mRNA, the COX-2 mRNA or cDNA derived therefrom is amplified in vitro. Applicable amplification assays are detailed in co-owned U.S. patent No. application Ser., as herein incorporated by reference, and include but are not limited to polymerase chain reaction (PCR); reverse transcriptase polymerase chain reaction (RT-PCR), ligase chain reaction, DNA signal amplification methods including branched chain signal amplification, amplifiable RNA reporters, Q-beta replication, transcription-based amplification, boomerang DNA amplification, strand displacement activation, cycling probe technology, isothermal nucleic acid sequence based amplification, and other self-sustained sequence replication assays.

In preferred embodiments of the methods of the invention, COX-2 mRNA is converted into cDNA using reverse transcriptase prior to in vitro amplification using methods known in the art. For example, a sample, such as 10 microL extracted serum RNA is reverse-transcribed in a 30 microL volume containing 200 Units of Moloney murine leukemia virus (MMLV) reverse transcriptase (Promega, Madison, Wis.), a reaction buffer supplied by the manufacturer, 1 mM dNTPs, 0.5 micrograms random hexamers, and 25 Units of RNAsin (Promega, Madison, Wis.). Reverse transcription is typically performed under an overlaid mineral oil layer to inhibit evaporation and incubated at room temperature for 10 minutes followed by incubation at 37° C. for one hour.

Alternatively, other methods well known in the art can be used to reverse transcribe COX-2 RNA to cDNA, such as the methods disclosed in Subbarayan et al. (2001, Cancer Res. 61: 2720-2726); Souza et al. (2000, Cancer Res. 60: 5767-5772); or Yoshimura et al. (2000, Cancer 89: 589-96) as provided in these references incorporated herein by reference in their entirety.

Amplification primers used are specific for amplifying COX-2-encoding nucleic acid. In a preferred embodiment, amplification is performed by RT-PCR, preferably as set forth in Hla and Neilson (1992, Proc. Natl. Acad. Sci. USA 89: 7384-7388), or Lim et al. (2001, Lab. Invest. 81: 349-360), incorporated herein by reference in their entirety. In these embodiments, preferred oligonucleotide primer sequences are as follows:

Primer 1: 5′-TTCAAATGAGATTGTGGGAAAATTGCT-3′ (sense; SEQ ID No. 1)

Primer 2: 5′-AGATCATCTCTGCCTGAGTATCTT-3′ (antisense; SEQ ID No. 2).

Amplification of COX-2 RNA yields a 305 bp PCR product fragment.

In an example of a preferred embodiment of the invention, COX-2 RNA is harvested from approximately 1.75 mL serum or plasma, and RNA extracted therefrom the Perfect RNA Total RNA Isolation Kit (Five Prime-Three Prime) according to manufacturer's directions. From this extracted RNA preparation, 10 microL are then reverse transcribed to cDNA as described above. RT-PCR for the COX-2 cDNA is performed using 5 microL of COX-2 cDNA in a final volume of 50 microL in a reaction mixture containing 1U of Amplitaq Gold (Perkin Elmer Corp., Foster City, Calif.), a reaction buffer provided by the Amplitaq supplier, 1.5 mM MgCl₂, 200 microM each dNTP, and 10 picomoles each of Primer 1 and Primer 2 identified above. The mixture is then amplified in a single-stage reaction in a thermocycler under a temperature profile consisting of an initial 2 minute incubation at 95° C., followed by 45 cycles of denaturation at 95° C. for 30 seconds, annealing at 60° C. for 30 seconds, and extension at 72° C. for 30 seconds, followed by a final extension at 72° C. for 5 minutes. Detection of the amplified product is then achieved, for example, by gel electrophoresis through a 4% Tris-borate-EDTA (TBE) agarose gel, using ethidium bromide staining for visualization and identification of the product fragment.

The invention also provides alternative methods of amplification of COX-2 RNA or cDNA known in the art, including but not limited to the methods of Souza et al. (2000, ibid.); Subbarayan et al. (2001, ibid.); and Yoshimura et al. (2000, ibid.), incorporated herein by reference in their entirety. Amplification methods can also be performed using primers specific for an internal control sequence, such as glyceraldehyde-3-phosphate dehydrogenase or beta-actin, as described in said references.

In a particularly preferred embodiment, COX-2 RNA or cDNA is amplified by RT-PCR in a quantitative amplification reaction. Preferred methods of quantitative amplification of COX-2 RNA are by the methods of Sales et al. (2001, J. Clin. Endocrinol. Metab. 86: 2243-2249), incorporated herein by reference in its entirety. Another particularly preferred method of quantitative amplification of COX-2 RNA or cDNA is the method of Agoff et al. (2000, Am. J. Pathol. 157: 737-745), incorporated herein by reference in its entirety. Quantitative amplification of COX-2 RNA or cDNA is particularly advantageous because this method enables statistically-based discrimination between patients with neoplastic disease and populations without neoplasm, including normal individuals, or with populations having arthritis or other inflammatory diseases. Using these methods, quantitative distributions of COX-2 RNA in bodily fluids such as blood plasma or serum are established for populations with neoplastic diseases, with arthritic or inflammatory diseases, and normal populations. Using this population information, the amount of extracellular COX-2 RNA in an individual is compared with the range of amounts of extracellular COX-2 RNA in said populations. This comparison results in a determination of whether the detected amount of extracellular COX-2 RNA in an individual indicates that the individual has a premalignant, neoplastic or malignant disease.

In alternative preferred embodiments, amplified products can be detected using other methods, including but not limited to gel electrophoresis; capillary electrophoresis; ELISA or modifications thereof, such as amplification using biotinylated or otherwise modified primers; nucleic acid hybridization using specific, detectably-labeled probes, such as fluorescent-, radioisotope-, or chromogenically-labeled probe; laser-induced fluorescence; Northern blot analysis; Southern blot analysis; electrochemiluminescence; reverse dot blot detection; and high-performance liquid chromatography. Furthermore, detection may be performed in either a qualitative or quantitative fashion.

PCR product fragments produced using the methods of the invention can be further cloned into recombinant DNA replication vectors using standard techniques (see Sambrook et al., 2001, MOLECULAR CLONING: A LABORATORY MANUAL, 3^{rd} ed., Cold Spring Harbor Laboratory, New York). RNA can be produced from cloned PCR products, and in some instances the RNA expressed thereby, using the TnT Quick Coupled Transcription/Translation kit (Promega, Madison, Wis.) as directed by the manufacturer.

The methods of the invention as described above can be performed in like manner for detecting extracellular COX-2 mRNA from other bodily fluids, including but not limited to whole blood, urine, effusions, ascitic fluid, saliva, cerebrospinal fluid, cervical secretions, vaginal secretions, endometrial secretions, gastrointestinal secretions, breast fluid or secretions, and bronchial secretions including sputum, and from washings or lavages. Although fractionation of the bodily fluid into its cellular and non-cellular components is not required for the practice of the invention, the non-cellular fraction may be separated, for example, by centrifugation or filtration of the bodily fluid.

The methods of the invention are thereby useful in the practice of diagnostic methods for detecting COX-2 mRNA over-expression in an animal, most preferably a human at risk for developing or who has developed a premalignant, neoplastic or malignant disease consisting of cells over-expressing COX-2 mRNA. The invention is particularly useful for evaluating individuals potentially at risk for neoplastic disease, wherein said individual has a familial history or a genetic predisposition of developing a malignancy or premalignancy. The invention further provides a method of identifying humans at risk for developing, or who have developed premalignancies or cancer, including but not limited to cancers of the breast, prostate, ovary, lung, cervix, colon, rectum, stomach, liver, pancreas, bladder, endometrium, kidney, skin including squamous cell cancer and malignant melanoma, and esophagus, as well as premalignancies and carcinoma in-situ including but not limited to prostatic intraepithelial neoplasia (PIN), cervical dysplasia and cervical intraepithelial neoplasia (CIN), bronchial dysplasia, a typical hyperplasia of the breast, ductal carcinoma in-situ, colorectal adenoma, a typical endometrial hyperplasia, and Barrett's esophagus.

In additional embodiments, the methods of the invention are useful as an aide in identifying or monitoring individuals having a non-neoplastic disease, such as arthritis or inflammatory disease, that over-express COX-2 and produce extracellular COX-2 RNA as a consequence or sequella thereof.

The diagnostic methods and advantageous applications of the invention can be performed using a diagnostic kit as provided by the invention, wherein the kit includes primers specific for COX-2 cDNA synthesis or in vitro amplification or both, and/or specific probes for detecting COX-2 RNA, cDNA or in vitro amplified DNA fragments thereof. The kit may further include instructions and reagents for extracting COX-2 RNA from a bodily fluid, wherein the bodily fluid includes but is not limited to plasma or serum, and/or reagents for the reverse transcription, amplification, or detection of COX-2 RNA or cDNA derived therefrom.

The inventive methods permit non-specific therapies, including anti-neoplastic therapies and non-selective inhibitors of cyclooxygenase such as aspirin and nonselective nonsteroidal anti-inflammatory drugs, as well as COX-2-selective or specific therapies, and combinations thereof, to be assigned and monitored in the treatment of diseases and disorders in animals, particularly humans. The invention in particular enables stratification and selection of patients likely to benefit from COX-2-directed therapy, including drugs or other specific therapies wherein COX-2 is inhibited or its actions blocked, such as specific COX-2 inhibitor drugs such as celecoxib and rofecoxib. The inventive methods allow therapeutic response to be monitored qualitatively or, thereby predicting relapse or providing a prognosis in COX-2 producing neoplastic and inflammatory diseases. In a particularly preferred embodiment, the invention can be used to determine that a COX-2-directed therapy is therapeutically indicated even in cases of premalignancy, early cancer, occult cancer or minimum residual disease. Thus, the invention permits selection of patients for said therapies or monitoring of therapeutic intervention, including chemoprevention, when tumor burden is low or when malignancy has not yet developed.

The invention further enables COX-2 RNA to be evaluated in blood plasma or serum or other bodily fluid in combination with detection of other tumor-associated or tumor-derived RNA or DNA in a concurrent or sequential fashion, such as in a multiplexed assay or in a chip-based assay, thereby increasing the sensitivity or efficacy of the assay in the detection or monitoring of neoplastic diseases, or in selecting an individual for a particular therapeutic regimen.

The methods of the invention and preferred uses for the methods of the invention are more fully illustrated in the following Examples. These Examples illustrate certain aspects of the above-described method and advantageous results. These Examples are shown by way of illustration and not by way of limitation.

### EXAMPLE 1

A 37 year-old man with a family history of colorectal cancer undergoes a cancer predisposition screening test by providing a blood plasma sample for a multiplexed assay that includes evaluation of the plasma for COX-2 RNA. COX-2 RNA is evaluated by the methods of the invention in a quantitative manner as described. In addition, other tumor-associated nucleic acids, including K-ras DNA and hTERT RNA, are evaluated by the multiplexed assay. The assay indicates COX-2 RNA is present in the plasma at levels higher than expected in the normal population. In addition, the multiplexed assay is positive for mutated K-ras oncogene present in the plasma, but negative for hTERT RNA. Overall, assay results indicate an increased predisposition for neoplasia. The man subsequently undergoes a conventional colonoscopy, and has two adenoma are detected and removed. As the patient is considered at high risk for developing colorectal neoplasia in the future, the man starts a chemopreventive drug therapy regimen consisting of a COX-2 inhibitor. Serial evaluation of quantitative COX-2 RNA levels in plasma is undertaken to evaluate response to the chemoprevention regimen. COX-2 RNA levels demonstrate progressive decline into the range for a normal population during the treatment period, indicating a good response to therapy.

This example demonstrates use of the invention for detection and monitoring of neoplasia, and determining predisposition to neoplasia. Futhermore, the example demonstrates use of the invention in monitoring response to a chemoprevention regimen that employs a COX-2 inhibitor drug.

### EXAMPLE 2

A 52 year-old woman with a long-standing history of fibrocystic breast disease is concerned about her risk for developing breast cancer. Although she receives yearly mammograms that have always been negative, the presence of the fibrocystic disease makes interpretation of the mammograms more difficult. The woman seeks her physician's advice regarding her risk for breast cancer, and possible chemopreventive therapy. The physician evaluates the patient by inserting a catheter into a breast duct, and aspirating and lavaging the duct. The aspiration fluid and lavage fluid is then sent for cytologic evaluation; and for analysis of COX-2 RNA using the methods of the invention in a qualitative manner. Cytology is negative. However, higher than normally expected levels of COX-2 RNA is detected in the aspiration and lavage fluids. The physician recommends that the woman continue to be followed closely, and initiates a chemoprevention regimen with a COX-2 inhibiting drug.

This example demonstrates the use of the invention to identify individuals who might benefit from COX-2 inhibitor therapies.

### EXAMPLE 3

A 64 year-old woman with metastatic non-small cell lung cancer is evaluated for a treatment regimen that is comprised of a combination of an anti-neoplastic cytotoxic agent with a COX-2 inhibitor agent, for example but not limitation, celecoxib with a taxane. Plasma COX-2 RNA levels, as determined by the inventive methods, will indicate that the woman's tumor is likely to over-express COX-2 RNA, and is therefore likely to benefit from the regimen.

This example demonstrates the use of the invention to identify individuals with cancer who might benefit from a therapeutic regimen comprising a COX-2 inhibitor drug.

It should be understood that the foregoing disclosure emphasizes certain specific embodiments of the invention and that all modifications or alternatives equivalent thereto are within the spirit and scope of the invention as set forth in the appended claims.

What is claimed is:

1. A method for detecting extracellular COX-2 RNA in blood plasma or serum, the method comprising the steps of:
a) extracting extracellular RNA from blood plasma or serum;
b) amplifying or signal amplifying a portion of the extracted extracellular RNA or cDNA prepared therefrom, wherein said fraction comprises extracellular COX-2 RNA, and wherein amplification is performed either qualitatively or quantitatively using primers or probes specific for COX-2 RNA or cDNA; and
c) detecting the amplified COX-2 RNA or cDNA product or signal.

2. A method of detecting extracellular COX-2 RNA in a bodily fluid, the method comprising the steps of:
a) extracting extracellular RNA from a bodily fluid;
b) amplifying or signal amplifying a fraction of the extracted extracellular RNA or cDNA prepared therefrom, wherein said fraction comprises extracellular COX-2 RNA, and wherein amplification is performed either qualitatively or quantitatively using primers for COX-2 RNA or cDNA; and
c) detecting the amplified COX-2 RNA or cDNA product fragment or signal.

3. The method of claim 2, wherein the bodily fluid is whole blood, blood plasma, serum, urine, effusions, ascites, saliva, cerebrospinal fluid, cervical secretions, endometrial secretions, gastrointestinal secretions, bronchial secretions, or breast fluid, lavages, or aspirations.

4. The method of claim 1, wherein the amplification in step (b) is performed by an RNA amplification method that amplifies the RNA directly or wherein the RNA is first reverse transcribed to cDNA, whereby the cDNA is amplified, wherein the amplification method is reverse transcriptase polymerase chain reaction, ligase chain reaction, DNA signal amplification, amplifiable RNA reporters, Q-beta replication, transcription-based amplification, isothermal nucleic acid sequence based amplification, self-sustained sequence replication assays, boomerang DNA amplification, strand displacement activation, or cycling probe technology.

5. The method of claim 2, wherein the amplification in step (b) is performed by an RNA amplification method that amplifies the RNA directly or wherein the RNA is first reverse transcribed to cDNA, whereby the cDNA is amplified, wherein the amplification method is reverse transcriptase polymerase chain reaction, ligase chain reaction, DNA signal amplification, amplifiable RNA reporters, Q-beta replication, transcription-based amplification, isothermal nucleic acid sequence based amplification, self-sustained sequence replication assays, boomerang DNA amplification, strand displacement activation, or cycling probe technology.

6. The method of claim 1, wherein detection of amplified product in step (c) is performed using a detection method that is gel electrophoresis, capillary electrophoresis, ELISA detection using biotinylated or otherwise modified primers, labeled fluorescent or chromogenic probes, laser-induced fluorescence, Northern blot analysis, Southern blot analysis, electrochemiluminescence, reverse dot blot detection, or high-performance liquid chromatography.

7. The method of claim 2, wherein detection of amplified product in step (c) is performed using a detection method that is gel electrophoresis, capillary electrophoresis, ELISA detection using biotinylated or otherwise modified primers, labeled fluorescent or chromogenic probes, laser-induced fluorescence, Northern blot analysis, Southern blot analysis, electrochemiluminescence, reverse dot blot detection, or high-performance liquid chromatography.

8. A method of identifying a human having COX-2 expressing cells or tissue, the method comprising the steps of:
a) extracting extracellular RNA from a non-cellular fraction of a bodily fluid;
b) amplifying or signal amplifying a fraction of the extracted extracellular RNA or cDNA prepared therefrom wherein said fraction comprises COX-2 RNA and wherein amplification is performed qualitatively or quantitatively using primers or probes specific for COX-2 RNA or cDNA; and
c) detecting the amplified COX-2 RNA or cDNA product fragment.

9. The method of claim 8, wherein the COX-2 expressing cells or tissue comprise a malignant or premalignant cell or tissue.

10. The method of claim 8, wherein the human has a familial history or a genetic predisposition of developing a malignancy or premalignancy.

11. The method of claim 8, wherein the human has a malignancy or premalignancy.

12. A method according to claim 1, wherein a human having a familial history or a genetic predisposition of malignancy or premalignancy is identified as having a premalignancy or malignancy when COX-2 RNA is detected in blood plasma or serum.

13. A method according to claim 2, wherein a human having a familial history or a genetic predisposition of malignancy or premalignancy is identified as having a premalignancy or malignancy when COX-2 RNA is detected in a bodily fluid of said human.

14. A method according to claim 1, whereby a cancer or premalignant condition is detected, identified, diagnosed, monitored, or evaluated.

15. A method according to claim 2, whereby a cancer or premalignant condition is detected, identified, diagnosed, monitored, or evaluated.

16. A method according to claim 1, whereby a non-neoplastic disease is monitored or evaluated.

17. A method according to claim 2, whereby a non-neoplastic disease is monitored or evaluated.

18. A method for preparing COX-2 cDNA, comprising the steps of extracting extracellular COX-2 RNA from blood plasma or serum and reverse transcribing the extracellular COX-2 RNA into COX-2 cDNA.

19. A method for preparing COX-2 cDNA, comprising the steps of extracting extracellular COX-2 RNA from a non-cellular fraction of a bodily fluid and reverse transcribing the extracellular COX-2 RNA into COX-2 cDNA.

20. A method for detecting extracellular COX-2 RNA, comprising the steps of extracting extracellular COX-2 RNA from blood plasma or serum, and hybridizing the RNA, or its corresponding cDNA derived therefrom, to a primer or probe specific for COX-2 RNA or its corresponding cDNA.

21. A method for detecting extracellular COX-2 RNA, comprising the steps of extracting extracellular COX-2 RNA from a non-cellular fraction of a bodily fluid, and hybridizing the RNA, or its corresponding cDNA derived therefrom, to a primer or probe specific for COX-2 RNA or its corresponding cDNA.

22. A method for treating an individual having COX-2 RNA in blood plasma or serum detected according to the method of claim 1, comprising the step of initiating or maintaining a COX-2 directed therapy in the individual.

23. A method for treating an individual having COX-2 RNA in blood plasma or serum detected according to the method of claim 2, comprising the step of initiating or maintaining a COX-2 directed therapy in the individual.

24. The method of claim 22, wherein the COX-2 directed therapy comprises administration of a COX-2 inhibitor.

25. The method of claim 23, wherein the COX-2 directed therapy comprises administration of a COX-2 inhibitor.

26. A method for monitoring an anti-COX-2 therapy, comprising the step of detecting COX-2 RNA in blood plasma or serum according to the method of claim 1.

27. A method for monitoring an anti-COX-2 therapy, comprising the step of detecting COX-2 RNA in blood plasma or serum according to the method of claim 2.

28. A diagnostic kit, comprising COX-2 specific amplification primers or probes and a reagent for extracting RNA from plasma or serum.
